(19) United States
(12) Patent Application Publication
(10) Pub. No.: US 2007/0225376 A1
(43) Pub. Date: Sep. 27, 2007

(54) Medicinal Agent

(76) Inventor: Mikhail Vladimirovich Kutushov, Moscow (RU)

Correspondence Address: PAULEY PETERSEN & ERICKSON, 2800 WEST HIGGINS ROAD, SUITE 365, HOFFMAN ESTATES, IL, 60195, US

(21) Appl. No.: 11/596,051

(22) PCT Filed: Jul. 13, 2004
(86) PCT No.: PCT/RU04/00272

§ 371 Date: Nov. 10, 2006

(30) Foreign Application Priority Data
May 12, 2004 (RU) 2004114297

(51) Int. Cl. A61K 31/136; A61K 31/14; A61P 35/00; C07C 211/45

(52) U.S. Cl. 514/647; 564/321

(57) ABSTRACT

Medicine and pharmacology, in particular a medicinal agent exhibiting anti-tumoral and immunomodulatory actions and including a tri-p-amino-thiphenyl-chlormethan tetramethylated, pentamethylated or hexamethylated derivative, the mixtures thereof or the combination thereof with dextrin. The agent of this invention exhibits a high activity and a reduced side effect.

### BACKGROUND OF THE INVENTION

1. Field of the Invention

This invention relates to the medical sector, more especially with anti-blastomatous medicines, and also with preparations that influence the immune system.

2. Discussion of Related Art

The preparation adriamycin is known and is an antibiotic of the anthracyline group with a marked anti-blastomatous effect (see M. D. Maschkowskij, Medicinal Agents, Medicine Publishers, Moscow, 1985, Volume 2, Pages 460-461).

The preparation is used in the form of a hydrochloride solution, exclusively intravenously as it causes subcutaneous tissue necrosis. Also, the preparation has a cardiotoxic effect and can cause cardiac pain, cardiac insufficiency and a lowering of the blood pressure.

The nearest prototype is cyclophosphan which is produced as a white crystalline powder that is water-soluble (1:50), lightly alcoholic and difficult to dissolve in an isotonic sodium chloride solution (for example, M. D. Maschowskij, Medicinal Agents, Medicine Publishers, Moscow, 1985, Volume 2, Pages 433-434.)

The known preparation has a wide, anti-blastomatous spectrum of activity and at the same time is an immunosuppressant, when such is used however, side effects can be observed, such as, for example, dizziness, nausea and vomiting, and often hair loss.

### SUMMARY OF THE INVENTION

It is one object of this invention to provide a medicine that is similar to the prototype but that has a wide spectrum of activity without side effects.

Methyl violet, which belongs to the group of triaminotriphenylmethane dyes, is known and provides salts with colored organic cations (see, for example, Abbreviated Chemical Encyclopaedia, Russian Encyclopaedia Publishers, Moscow, 1967, Volume 5, Pages 252-253) and provides a mixture of nitrogen methylised tri-p-aminotriphenylchloromethane that includes tetra methyl, penta methyl and hexamethyl derivatives. (See for example, StainsFile methyl violet 2B, 6B and 10B.) (http://members.pgonline.com/bryand/StainsFile/dyes/42535.htm).

The structural formula of tri-p-aminotriphenylchloromethane has the following form: 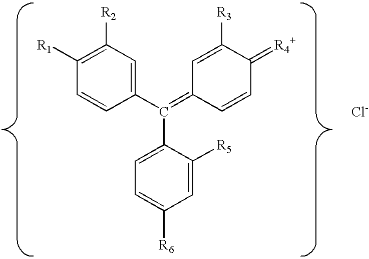
in which, for example, R₁═N(CH₃)₂, R₂═H, R₃═H, R₄═N(CH₃)₂, R₅═H, R₆=either NH₂ or NHCH₃ or N(CH₃)₂.

Methyl violet 2B (tetramethyl pararosanilin chloride), in this case, is a tetramethyl derivative of tri-p-aminotriphenylchloromethane (in the structural formula R₆═NH₂, designations for R₁, R₂, R₃, R₄ and R₅ correspond to the abovementioned, total formula C₂₃H₂₆N₃CI, molecular weight 379.94) and is a water-soluble and alcohol-soluble, fine-grained, brown, crystalline powder with a hint of green. Methyl violet 6B (pentamethyl pararosanilin chloride) is a pentamethyl derivative of tri-p-aminotriphenylchloromethane (in the structural formula R₅═NHCH₃, designations for R₁, R₂, R₃, R₄ and R₅ correspond to the abovementioned, total formula C₂₄H₂₈N₃CI, molecular weight 393.97) and is a homogeneous, water-soluble and alcohol-soluble, fine-grained, crystalline powder in a bronze-green color, also known as basic violet K or respectively methyl violet. Methyl violet 10B (hexamethyl pararosanilin chloride) is a hexamethyl derivative of tri-p-aminotriphenylchloromethane (in the structural formula R₆═N(CH₃)₂, designations for R₁, R₂, R₃, R₄ and R₅ correspond to the above-mentioned, total formula C₂₅H₃₀N₃CI, molecular weight 407.99) and is a water-soluble and alcohol-soluble, green, crystalline powder, also known as crystal violet or respectively basic violet 3 (see, for example, Catalogue of Harmful Substances in Industry, Part 1, Organic Substances, Chemical Literal Publishers, Edition 4, Leningrad, 1963, Pages 638-639, StainsFile Methyl Violet 2B, 6B and 10B.) (http://members.pgonline.com/bryand/StainsFile/dyes/42535.htm) and Reference Work for Chemists, Chemistry Publishers, Leningrad, 1967, Volume 6, Page 759). The above-mentioned derivatives are designated in some sources as gentian violet (see, for example, StainsFile Methyl Violet 2B, 6B and 10B) (http://members.pgonline.com/bryand/StainsFile/dyes/42535.htm).

Known is a dye mixture, including comprising methyl violet and fuchsine under the designation dahlia violet, a water-soluble, alcohol-soluble, green, crystalline powder, and comprising methyl violet and dextrin under the designation gentian violet, which is a water-soluble, bright green powder (see, for example, Catalogue of Harmful Substances in Industry, Part 1, Organic Substances, Chemical Literature Publishers, Edition 4, Leningrad, 1963, Page 639).

These dyes are mainly used in the industry, for example to color tissue. The use of crystal violet (hexamethyl derivative of tri-p-aminotriphenylchloromethane), however, in gynaecology and paediatrics is known to treat candidid (see http://www.oncology.com).

A dextrin, which is a polysaccharide mixture, is known and used in tissue coloring (see N. L. Glinka, General Chemistry, Chemistry Publishers, 1978, Page 494).

The above object is achieved through the intermediary of an immuno-modulating, anti-blastomatous medicine, in which the medicine includes derivatives of tri-p-aminotriphenylchloromethane that can be represented pharmacologically, and the medicine has the following structural formula: 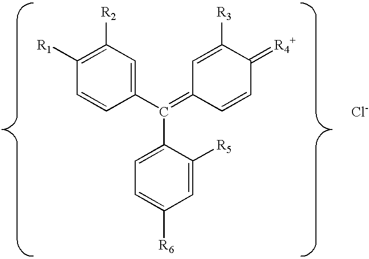
wherein, for example, the following applies: R₁═N(CH₃)₂, R₂═H, R₃═H, R₄═N(CH₃)₂, R₅═H, a R₆=either NH₂ or NHCH₃ or N(CH₃)₂.

The medicine, in this case, includes the tri-p-aminotriphenylchloromethane in the form of either its tetramethyl derivative, in the structural formula R₆═NH₂, or its pentamethyl derivative, in the structural formula R₆═NHCH₃, or its hexamethyl derivative, in the structural formula R₆═N(CH₃)₂, wherein the designations of the named derivatives R₁, R₂, R₃, R₄ and R₅ correspond to the above-mentioned.

Also, the medicine includes the tri-p-aminotriphenylchloromethane either as a mixture of its tetramethyl and pentamethyl derivatives in the ratio of 2.0-98.0 percent by volume tetramethyl derivative and the remainder pentamethyl derivative or as a mixture of its tetramethyl and hexamethyl derivatives in the ratio of 2.0-98.0 percent by volume tetramethyl derivative and the remainder hexamethyl derivative or as a mixture of its pentamethyl and hexamethyl derivatives in the ratio of 2.0-98.0 percent by volume pentamethyl derivative and the remainder hexamethyl derivative or as a mixture of its tetramethyl, pentamethyl and hexamethyl derivatives in the ratio of 1.5-97.0 percent by volume tetramethyl derivative, 1.5-97.0 percent by volume pentamethyl derivative and the remainder hexamethyl derivative.

### DETAILED DESCRIPTION OF THE INVENTION

The above object is achieved through the intermediary of an immuno-modulating, anti-blastomatous medicine wherein the medicine comprises a mixture of dextrin and pharmacologically representable derivatives of the tri-p-aminotriphenylchloromethane, for example, in the form of either tetramethyl or pentamethyl or hexamethyl derivatives or respectively a mixture of tetramethyl and pentamethyl derivatives or a mixture of tetramethyl and hexamethyl derivatives or a mixture of pentamethyl and hexamethyl derivatives or respectively a mixture of tetramethyl, pentamethyl and hexamethyl derivatives in the ratio of 10.0-95.5 percent by volume of the corresponding derivative or respectively derivative mixture of the tri-p-aminotriphenylchloromethane and the rest dextrin.

In this case, the mixture of tetramethyl and pentamethyl derivatives of the tri-p-aminotriphenylchloromethane is selected at a ratio of 2.0-98.0 percent by volume tetramethyl derivative and the rest pentamethyl derivative. The mixture of tetramethyl and hexamethyl derivatives is selected at a ratio of 2.0-98.0 percent by volume tetramethyl derivative and the rest hexamethyl derivative. The mixture of pentamethyl and hexamethyl derivatives is selected at a ratio of 2.0-98.0 percent by volume pentamethyl derivative and the rest hexamethyl derivative. The mixture of tetramethyl, pentamethyl and hexamethyl derivative is selected at a ratio of 1.5-97.0 percent by volume tetramethyl derivative, 1.5-97.0 percent by volume pentamethyl derivative and the rest hexamethyl derivative.

The medicine with an immuno-modulating and anti-blastomatous effect contains a pharmacologically representable tetramethyl or pentamethyl or respectively hexamethyl derivative of the tri-p-aminotriphenylchloromethane or of its mixtures, or respectively mixture produced from one derivative and dextrin or mixtures produced from the derivatives and dextrin.

The tetramethyl derivative of the tri-p-aminotriphenylchloromethane, in this case, is methyl violet 2B (tetramethyl pararosanilin chloride, in the structural formula R₆═NH₂, the designations for R₁, R₂, R₃, R₄ and R₅ correspond with the abovementioned, the total formula is C₂₃H₂₆N₃CI, molecular weight is 379.94), which is a water-soluble and alcohol-soluble, fine-grained, brown, crystalline powder with a hint of green. The pentamethyl derivative is methyl violet 6B (pentamethyl pararosanilin chloride, in the structural formula R₆═NHCH₃, the designations for R₁, R₂, R₃, R₄ and R₅ correspond to the above-mentioned, the total formula is C₂₄H₂₈N₃CI, the molecular weight is 393.97), which is a homogeneous, water-soluble and alcohol-soluble, fine-grained, crystalline powder that is bronze-green in color. The hexamethyl derivative is methyl violet 10B (hexamethyl pararosanilin chloride, in the structural formula R₆═N(CH₃)₂, the designations for R₁, R₂, R₃, R₄ and R₅ correspond to the abovementioned, the total formula is C₂₅H₃₀N₃CI, the molecular weight is 407.99), which is a water-soluble and alcohol-soluble, green, crystalline powder.

Mixtures from derivatives of the tri-p-aminotriphenylchloromethane and mixtures of its derivatives and dextrin in the above-mentioned ratios provide a water-soluble, greenish powder.

Derivatives of the tri-p-aminotriphenylchloromethane in the form of its mixtures are obtained by subsequent separation as tetramethyl, pentamethyl and hexamethyl derivatives by methylizing parafuchsin with iodomethyl (see A. N. Nesmejanow, N. A. Nesmejanow, Basics of Organic Chemistry, Chemistry Publishers, Moscow, 1974, Volume 2, Page 200), wherein the pentamethyl derivative (basic violet K) can also be obtained by oxidizing dimethylaniline with copper salts (see Harmful Substances in Industry, Part 1, Organic Substances, Chemical Literature Publishers, Edition 4, Leningrad, 1963, Page 639) and pure hexamethyl derivative (crystal violet) can be obtained by condensation of di-(p-dimethylamino)-benzophenone (Michler's ketone) with dimethylaniline and subsequent acidifying (see A. N. Nesmejanow, N. A. Nesmejanow, Basics of Organic Chemistry, Chemistry Publishers, Moscow, 1974, Volume 2, Page 200).

The target product in the form of a medicine that is ready for use is obtained by purifying the mixtures obtained, for example by re-crystallization as well as by using chromatography by passing water solutions of these salts through an adsorption layer, for example chromatographic protoplasm or bentonite clay in the separation column.

Dextrin is obtained by heating dry starch to 200-250° C. (see N. L. Ginka, General Chemistry, Chemistry Publishers, 1978, Page 494).

The required mixtures are obtained by selecting specific substances at a predetermined ratio.

The medicine containing pharmacologically representable derivatives of tri-p-aminotriphenylchloromethane as well as the mixtures of dextrin and the derivatives have an anti-blastomatous effect, which, is accompanied, for example, by the therapeutic effect that has been established by the inventor, the effect being designated as disymmetrical and being measured at different temperatures at different parts of the body. This effect can be seen, for example, in a temperature range of 2-3° C. 0.5-2 hours after the medicine has been taken and lasts for 3-4 hours (see M. W. Kutuschow, Cancer, Healing is Possible, Newa Publishers, St-Petersburg, 2003). The medicine also has an anabolic activity and promotes cell and humeral protection factors of the body.

The method of activity of the pharmacologically representable derivations of the medicine containing tri-p-aminotriphenylchloromethane is based on ionizability, through which interaction with phospholipids of the cell membranes occurs to disturb the balance of the ionic trans-membrane potential, cell death thereby resulting. The investigations carried out by the inventor also showed that that medicine is an inhibitor of the mitochondrial ferments of malicious cells and causes the mitochondria of the carcinoma cells to die, suppressing the NADPH cytochrome c reductase activity, thereby blocking energy (see Shumyantseva V. V., Uvarov V. Yu., Byakova O. E., Archakov A. I., Biochem. Molec. Biol. Intern, 1996, No. 38, Pages 829-838). It is established that the effectiveness of the medicine is all the greater, the more its adsorption spectrum is pushed in the direction of the UV range. In addition, it is established that dextrin as one of the substances considerably reduces the toxicity of the medicine and consequently expands the dosing range.

This has been confirmed through laboratory tests and patient tests, the blood being tested for the immunological values of the white blood cell and lymph systems.

Produced as technically or chemically pure salt or respectively highly purified or medically purified or medically highly purified salt, in accordance with pharmacopeia requirements on medicines (see Law of the Russian Federation on Medicines No. 86-FZ dated Jun. 22, 1998 and the branch standard OST 91500.05.001,00, Quality Standards for Medicines, Conditions, publication in the Rossijskaja Gaseta dated Nov. 28, 2001), the medicine can be given to the patient, with reference to the degree of purification of the preparation, in an isotonic sodium chloride solution intravenously and perorally in capsules as well as in the form of a substance in ointments or as medical enemas or respectively suppositories, the use of the medicine not causing any allergic or other side effects and its effectiveness (in the treatment of certain illnesses) in some cases being greater than that of the prototype.

The results of tests on the working mechanism of the medicinal agent in accordance with this invention are detailed in the examples 1-3 below, and the possible use of the medicinal agent is confirmed by the examples 4-10 below.

### EXAMPLE 1

In test glasses (14×10 ml test glasses) of the first group with human malignant melanoma A 375 in buffer solution, the medicinal agent was introduced in a dilution of 10⁻⁷ mmol/l in the form of methyl violet 2B (tetramethyl derivative of the tri-p-aminotriphenylchloromethane), methyl violet 6B (pentamethyl derivative of the tri-p-aminotriphenylchloromethane), methyl violet 10B (hexamethyl derivative of the tri-p-aminotriphenylchloromethane), mixtures of methyl violet 2B and methyl violet 6B, methyl violet 2B and methyl violet 10B, methyl violet 6B and methyl violet 10B, mixtures of methyl violet 2B, methyl violet 6B and methyl violet 10B (the substances at identical ratios), mixtures of dextrin and its salts as well as mixtures of dextrin and the specified mixtures of these salts (at a ratio of 50 percent by volume dextrin and the rest salt or the rest salt mixture (at the specified ratio)). The same was carried out with a dilution of 10⁻⁹mol/l (14×10 ml test glasses).

In test glasses of the second group with HFF (human forskin fibroblast), the medicine was introduced in the same compositions and dilutions.

In addition, a third control group of test glasses (6 glasses) was formed with the same cancer cell pool and fibroblasts, into which adriamycin and cyclophosphan in the same dilution was introduced, as well as a corresponding test glass group with a physiological salt solution.

After thermostatization of the above-mentioned test glasses for a week at 37° C. (in the dark), the contents were analyzed.

The results obtained were as follows.

In the test group with the medicine in a dilution of 10⁻⁷ mmol/l, the medicine suppresses the replication of the melanoma cells by 86-93% in comparison to the replication of these cells in the test glass with a physiological salt solution and suppresses the replication of the fibroblasts by 25-27%, the phenotype being approximately identical for all compositions of the medicine.

In test glasses of the control group with adriamycin, a replication of the melanoma cells was reduced by 42% and with cyclophosphan it was reduced by 55%.

In the test group with the medicine in a dilution of 10⁻⁹ mmol/l, the medicine suppresses the replication of the melanoma cells by 75-90% in comparison to the replication of these cells in a physiological salt solution and suppresses the replication of the fibroblasts by 5-10%, the replication of the melanoma cells being reduced by 30% in test glasses of the control group with adriamycin and by 35% in test glasses with cyclophosphan.

The data obtained points to the fact that the medicine put forward, even in very strong dilutions, has a marked anti-blastomatous effect and greater activity than known anti-blastomatous preparations and is characterized by a small cytotoxic effect against fibroblasts.

### EXAMPLE 2

In test glasses (50×10 ml test glass) of the test group with “smooth” microsomes of cancer cells MCF-7 (mammary gland adeno carcinoma) in a buffer solution, the medicinal agent was introduced in a dilution of 10⁻⁵ mmol/l in the form of mixtures of methyl violet 2B (tetramethyl derivative of the tri-p-aminotriphenylchloromethane) and methyl violet 6B (pentamethyl derivative of the tri-p-aminotriphenylchloromethane) in two ratios: 98.0 percent by volume methyl violet and the rest methyl violet 6B and 98.0 percent by volume methyl violet 6B and the rest methyl violet 2B, methyl violet 2B and methyl violet 10B (hexamethyl derivative of the tri-p-aminotriphenylchloromethane) in 2 ratios: 98 percent by volume methyl violet 2B and the rest methyl violet 10B, of methyl violet 6B and methyl violet 10B in two ratios: 98.0 percent by volume methyl violet 6B and the rest methyl violet 10B and 98.0 methyl violet 10B and the rest methyl violet 6B, mixtures of methyl violet 2B, methyl violet 6b and methyl violet 10B in four ratios: 1) 2.0 percent by volume methyl violet 2B, 50.0 percent by volume methyl violet 6B and the rest methyl violet 10B, 2) 50.0 percent by volume methyl violet 2B, 2.0 percent by volume methyl violet 6B and the rest methyl violet 10B, 3) 50.00 percent by volume methyl violet 6B, 2.0 percent by volume methyl violet 10B and the rest methyl violet 2B, 4) 20.0 percent by volume methyl violet 2B, 25.0 percent by volume methyl violet 6B and the rest methyl violet 10B and mixtures of dextrin and salts and the specified mixtures of these salts (in the abovementioned compositions) at the ratio: 10.0, 25.0, 75.0, 90.0 percent by volume dextrin and the rest salt or respectively salt mixture. The same was also implemented with a dilution of 10⁻⁹ mmol/l.

In test glasses (10 ml) of the second group with microsomes of rat liver, the medicine was introduced in the same compositions and dilutions.

In addition, a third (control) group of test glasses (6) was formed with microsomes of the same cancer cell pool and rat liver, into which adriamycin and cyclophosphan were introduced in the same dilution, and a corresponding test glass group with physiological salt solution.

In the test glasses of the groups the concentration of cytochrome C was determined, which is 50 μM for cancer microsomes and 20 μM for microsomes of rat liver.

After the thermostatization of the above-mentioned test glasses over 20 min at 25° C. (in the dark), the contents were analyzed.

The results obtained were as follows.

In the test group, the concentration of cytochrome C in the test glasses with the introduced salt mixture was determined in the range up to 60 μM and in the test glasses with the introduced mixtures of salt and dextrin it was in the range of up to 65 μM.

In the test glasses with adriamycin and cyclophosphan, the concentration of cytochrome C was 40-45 μM. In the test glass with the physiological salt solution no changes in the concentration of cytochrome C were observed. Liver microsomes of rats were not influenced by the preparation. The concentration of cytochrome C in all test glasses remained in the region of 19-20 μM.

The results obtained lead to the assumption that the medicine promotes the releasing of the “aggressive” protein of cytochrome C from the membranes of the cancer cell mitochondria, which causes an apoptosis of these cells, triggering a destruction mechanism of the DNS through caspases (see Wilson B. E., Mochon E. A., Boxer L. M., Induction of blc-2 expression by phosphorylated CREB proteins during B-cell activation and rescue from apoptosis, Mol. Cell. Biol., 1996, V. 16, Pages 5546-5556).

### EXAMPLE 3

A reaction to the medicinal agent in the form of salts of methyl violet 2B, methyl violet 6B and methyl violet 10B as well as mixtures of these salts with dextrin was tested in B-57 type mice in two ratios: 5.0 percent by volume dextrin and the rest salt as well as 95.0 percent by volume dextrin and the rest salt.

The test group included 60 mice and was divided into 6 sub groups each of 10 mice. The control group included 10 mice; the sub groups of the test group, in this case, were kept separate from the control group, and mice in each sub group were identified from 1 to 10 with a dye, in this case with Viride Nitens.

The test group received the medicinal agent over 10 days: the first sub group received the mixture produced from 2.0 percent by volume dextrin and the rest methyl violet 2B, the second sub group received the mixture from 90.0 percent by volume dextrin and the rest methyl violet 2B, the third sub group the mixture from 5.0 percent by volume dextrin and the rest methyl violet 6B, the fourth sub group received the mixture from 90.0 percent by volume dextrin and the rest methyl violet 6B, the fifth sub group received the mixture from 5.0 percent by volume dextrin and the rest methyl violet 10B and the sixth sub group received the mixture from 90.0 percent by volume dextrin and the rest methyl violet 10B. In each sub group, mice Nos. 1 to 8 received the preparation as a drink (in a water solution) and mice Nos. 9-10 received it as an injection into the belly fur. The single dose for mice Nos. 1-2 was 2 mg, for Nos. 3-4 5 mg, for Nos. 5-6 10 mg, for Nos. 7-8 20 mg and Nos. 9-10 15 mg per 1 ml injection solution. The mice in the control group did not receive any preparation.

Results: one week after the introduction of the medicinal agent no differences in the behavior of the even sub groups and the control group. Animals Nos. 5-6 in the uneven sub groups could hardly move, animals Nos. 7-8 in the uneven sub groups died.

The mice with uneven numbers in all the sub groups were killed on the 10^{th} day and mice with even numbers were killed on the 21^{st} day.

Traces of the medicinal agent were established in the kidneys of the animals Nos. 3-6 in the uneven sub groups and of the animals Nos. 5-8 in the even groups.

A histological test established a minimum influence of the mixture with methyl violet 10B (identified as B.V.2) and a maximum influence of the mixture with methyl violet 2B.

It can be concluded, taking the ratios of mass into account, that the single dose of 2.0 g is representable for the human body. More accurate data, however, can only be supplied after corresponding tests.

### EXAMPLE 4

B57 type mice in the test and control group (60 mice each) were injected with melanoma 16. The test group were divided into 6 sub groups and the control group was divided into 3 sub groups, each with 10 mice. Each sub group in the test group received 5 mg medicinal agent in the form of a water solution of methyl violet 2B, methyl violet 6B and methyl violet 10B as well as mixtures of these salts with dextrin at a ratio of 25:75 percent by volume.

The first and second sub group in the control group received a dose of 5 mg in a water solution of adriamycin and cyclophosphan in the form of a drink; the third sub group did not receive any preparation.

Results: On the 39^{th} day of the test, 39 of the 60 mice in the test group remained alive. 11 mice (4 from the first sub group and 3 from each of the second and third sub groups and one mouse from the fourth sub group) died on the 19^{th} day. 5 mice (2 from each of the first and second sub groups and one from the third sub group) died on the 23^{rd} day and 5 mice (2 from the first and one each from the second, fourth and fifth sub groups) died on the 34^{th} day of the test. Seventeen surviving mice (one from the first, three from the second, one from the third, 4 each from the fourth and fifth and 3 from the sixth sub group) had blastomas of ca. 0.2×0.2 mm; no blastoma was established on visual examination of the remaining mice. The surviving mice were killed on the 36^{th} day of the test. In the dissection, 9 mice (one from the first, 4 from the fourth and 2 each from the fifth and sixth sub groups) were found to have isolated, pulmonary metastasis and 4 of the said 9 mice (one from the first, 2 from the second and one from the fifth sub group) also had hepatic metastasis. The other mice had no metastasis and no visible changes of the inner organs.

The first and second sub group of the control group: 2 mice in the first sub group and 5 mice in the second sub groups survived as far as the 36^{th} day of the test. The blastomas had average dimensions of 1.5×1.5 cm. During the dissection, lung and liver injuries were established.

All the mice in the third sub group died on the 10^{th}, 12^{th}, 20^{th} and 25^{th} day. On investigation, bleeding blastomas of the back with an average size of 2.5×2.0 cm were established. A histological test established total lysis of the lung.

Conclusions: the medicinal agent has an anti-blastomatous effect and does not cause any side effects.

### EXAMPLE 5

Non-Hodgkin's lymphoma (NHL) was diagnosed in a 3-year old dog weighing 16 kg. The condition was serious, difficulty in breathing and cachexia. In the area of the neck there were growths to the size of 7×5 cm on the right and 11×10 on the left. Several growth-like images in the mediastinum were visible on the X-ray that was taken on 20.10.99.

Treatment with the medicinal agent was started on 30.10.99. The dog was given 500 mg methyl violet 2B per 500 ml physiological salt solution intravenously in the form of drops. After three days a mixture of methyl violet 2B and methyl violet 6B (250 mg each) per 500 ml physiological salt solution was given intravenously and a peroral dose of a mixture of dextrin, methyl violet 2B, methyl violet 6B and methyl violet 10B (50 mg of each substance in a single dose of 200 mg) in the form of powder twice a day. The mixture of methyl violet 2B and methyl violet 6B was given repeatedly intravenously as well as a peroral dose of the above-mentioned mixture with dextrin after one week.

Two weeks after the start of the treatment, an improvement was observed in the general condition and in the biochemical and clinical blood parameters. After three weeks, the visible growths were 2.5×smaller.

The treatment with methyl violet 2B and methyl violet 6B comprised a weekly intravenous intromission of the mixture for 3 months; the peroral dose of the abovementioned mixture (with dextrin) was reduced daily.

In an examination after the treatment, the dog proved to be in good condition, its weight was 19.5 kg, and there were no growths on the neck. There was no pathology in the blood results. Isolated small lung metastasis could be seen in the X-ray taken on 02.02.2000.

### EXAMPLE 6

A sarcoma of the right forearm and pulmonary metastases were diagnosed on a dog aged 6 years, weighing 21 kg. The x-ray that was taken on 10.07.2002 showed a breakdown of the periosteum and of the 1^{st} to 7^{th} right-hand ribs, several pulmonary metastases on the right and left and a growth of 10×15×12 cm.

On 12.07.02 treatment was started. Every other day a dose made up of 50 mg methyl violet 10B (identified as B.V.2), diluted with 9% NaCl and dissolved in 250 ml water was administered intravenously in an amount of 2 ml; paste bandages were wrapped around the growth daily (2% methyl violet 10B and the remainder baby lotion).

Three weeks after the start of the treatment, the dimensions of the growth had reduced to 7×7×3 cm. The condition was stable and after a further two weeks it was satisfactory; the X-ray showed an encapsulated growth of 2×2,5×1 cm and traces of rib and lung metastases.

### EXAMPLE 7

Exstirpation of the uterus and the surrounding area on account of cancer of the right ovary was performed on patient I who was 35 years old and had visited the doctor in November 2002. The tomogram that was dated 28.08.02 showed a recidiv, a growth of 8.8×9.8×14 cm growing through the urinary bladder and the rectum.

The patient was treated with an enema of the rectum: over five days once a day, after five procedures once every other day and thereafter every third day for 15 enemas. She was treated with a single dose of 20 mg medicinal agent (B.V.2) per 15-20 ml water, diluted in 2 ml 70% ethyl alcohol.

After 2 weeks of the treatment, ultrasound showed the size of the growth to be 3.8×4.2×3.1 cm with smooth contours; the growth had receded from the wall of the rectum, and the deformation of the urinary bladder wall was slight.

After the treatment, the growth according to an ultrasound test was encapsulated and was 1.3×2.0×1.6 cm in size; the capsule walls were 0.5 cm thick and there were no problems. Continuation of the treatment with intervals (1.0-1.5 months) was recommended. In a control investigation using ultrasound on 02.03.04, the growth was no longer to be found.

The blood parameters before treatment were: leucocytes up to 10,000, erythrocytes 2.5 million and thrombocytes 110,000. The blood parameters after the treatment were: leucocytes 9,000, erythrocytes 5.5 million and thrombocytes 300,000.

### EXAMPLE 8

A papillary thyroid cancer was diagnosed in 62 year old patient R. Before the treatment, the growth according to an ultrasound investigation was in the right lobe and was 5×3×1.7 cm in size.

The patient was treated with the medicinal agent (B.V.2) for 2 weeks; one gelatine capsule two times a day with 20 mg medicinal agent perorally after food and enemas in a single dose of 100 ml solution, comprising 50 mg medicinal agent powder, diluted in 2 ml 70% ethyl alcohol and dissolved in 100 ml water solution with 0.9% NaCl.

Over 7 days, 3 hours after taking the dose of the medicinal agent the patient was observed to be under a dissymmetric effect, which was manifested through sudden signs of fever and different temperatures on the right and left sides of the body, from 35.4-36.9° C. on the right and 37.3-38.6° C. on the left, over 1.0-1.5 hours. The above-mentioned temperature differences disappeared almost completely when the preparation was taken again.

After the treatment, an ultrasound investigation showed a primary growth and no metastases. In a control test after 2 months, the condition was satisfactory, and there were no complications.

### EXAMPLE 9

Patient Sch, aged 18, was diagnosed in September 2002 with a tumour of 2×2, 5×2 cm in her right breast. The mammogram showed a fibrocystic mastopathy in the right breast. A biopsy sample showed atypical cells.

The breast was painted two or three times in the area of the growth with a mixture of B.V.2 powder and baby lotion at a ratio of 10:90%. Repeated biopsy samples after 1.5 months showed no atypical cells. The growth could practically no longer be felt.

### EXAMPLE 10

An adeno carcinoma of the lower colon with liver metastases was diagnosed on patient Ju, aged 48 years. In April 1999, a colon was cut out and a colostomy put in. Prophylactic chemotherapy was not effective (renewed pain in the stomach area, periodic vomiting, general weakness). In August 2002, the patient went back to his doctor on account of these complications. A tomogram dated 21.09.02 showed a growth-like formation of 7.0×5.5 cm in the area of the spleen, uneven contours and metastases in the right liver lobes.

The following treatment was carried out: 10 days of peroral doses of a mixture of dextrin and methyl violet 2B, methyl violet 6B and methyl violet 10B (B.V.2) diluted with 100 ml water (at a corresponding ratio of 25:30:30:15) 1.0 g twice a day 30 minutes before food as well as in the mornings and evenings 0.5 g B.V.2, dissolved in 50 ml water, into the colostomy. Over and above this, with the agreement of the patient, an intravenous intromission of 500 ml of a 1% solution of the preparation to increase the effect. The treatment lasted for 3 months. After the treatment, the condition was satisfactory. The tomogram showed no growths in the abdomen and no hepatic metastases. The blood parameters were in order.

### EXAMPLE 11

When patient N, 25 years old, went to the doctor on 23.10.1999, she was diagnosed with complications of alopecia totalis, periodic weakness and depression.

Alopecia began with eye brow and eye lash hair loss 6 years ago after the flu, and then 6 months later with head hair loss. The illness was treated for 2 years locally with hormones and ointments. At an anamnesis it was established that the patient had had acute respiratory disease at the age of 7 and a week later had caught chickenpox, probably giving rise to a latent auto-immune disease. An auto-immune disease with complicated alopecia totalis was diagnosed. Blood tests for cellular and humeral immunity confirmed the diagnosis.

Two treatments were carried out using the medicinal agent. The first treatment was a dose of a mixture of dextrin, methyl violet 2B, methyl violet 6B and methyl violet 10B at a ratio of 25:25:25:25 at a regime of 0.2 ml 1% water solution twice a day 30 minutes before food over 3 months.

After 3 weeks of the treatment, gnawing pain appeared in the kidneys. This was assumed to be a reaction of the adrenal glands to the suppression of fixed, hyperactive microphages caused by the preparation. A tomogram of the abdomen, kidneys and adrenal glands showed that the left and right adrenal glands were larger and deformed. The treatment was continued and after one week the pain in the kidneys disappeared. After 2 months of the treatment, healthy hair appeared on her head and on her legs.

After the first batch of treatment, the treatment was interrupted for 2 months and then an additional batch of treatment of 2 months was carried out, which consisted of one dose of methyl violet 10B (B.V.2) with an amount of 0.5 ml in a 1% water solution twice a day 30 minutes before food.

One year after the start of the treatment, the thickness and structure of the body hair is typical. At the moment no hair loss is observed. The control tomogram dated 12.03.03 showed that the adrenal glands were not enlarged and were in the form of a triangle. The blood parameters were in order.

The following table shows the test results with reference to the cellular and humeral immunity:

| | Before treatment | After 4 weeks | After 6 months |
|---|---|---|---|
| Immunoglobulin A | 60 | 170 | 249 |
| Immunoglobulin M | 50 | 98 | 190 |
| Immunoglobulin G | 800 | 1400 | 1750 |
| T-lymphocytes, % | 34.0 | 65.0 | 74.0 |
| B-lymphocytes, % | 10.0 | 14.0 | 16.0 |
| Phagocytosis of | 43.0 | 60.0 | 67.3 |
| latex particles, % | | | |
| TNF | 15.6 | 23.0 | 32.5 |
| Helper cells, % | 26.0 | 30.0 | 38.0 |
| Suppressor cells, % | 12.0 | 18.0 | 17.0 |
| Killer cells, % | 16.0 | 19.0 | 19.0 |

1. A medicinal agent having an anti-blastomatous and immuno-modulating effect, comprising: the medicinal agent including derivatives of tri-p-aminotriphenylchloromethane that can be represented pharmacologically, the medicinal agent having a structural formula: 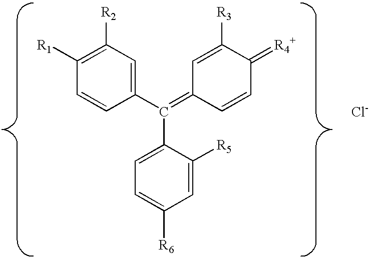
wherein at least the following applies: R₁═N(CH₃)₂, R₂═H, R₃═H, R₄═N(CH₃)₂, R₅═H, R₆=either NH₂ or NHCH₃ or N(CH₃)₂.

2. The medicinal agent according to claim 1, wherein the medicinal agent includes tri-p-aminotriphenylchloromethane in a form of a tetramethyl derivative, in a structural formula of which R₆═NH₂ and R₁, R₂, R₃, R₄ and R₅ correspond to one of named elements and respectively compounds.

3. The medicinal agent according to claim 1, wherein the medicinal agent includes tri-p-aminotriphenylchloromethane in the a form of a pentamethyl derivative, in a structural formula of which R₆═NHCH₃ and R₁, R₂, R₃, R₄ and R₅ correspond to one of named elements and respectively compounds.

4. The medicinal agent according to claim 1, wherein the medicinal agent includes tri-p-aminotriphenylchloromethane in a the form of a hexamethyl derivative, in a structural formula of which R₆═N(CH₃)₂ and R₁, R₂, R₃, R₄ and R₅ correspond to one of named elements and respectively compounds.

5. The medicinal agent according to claim 3, wherein the medicinal agent includes tri-p-aminotriphenylchloromethane in a form of a mixture, the tetramethyl and pentamethyl derivatives of which being at a ratio of 2-98 percent by volume tetramethyl derivative and a remainder pentamethyl derivative.

6. The medicinal agent according to claim 3, wherein the medicinal agent includes tri-p-aminotriphenylchloromethane in a form of a mixture, the tetramethyl and hexamethyl derivatives being at a ratio of 2-98 percent by volume tetramethyl derivative and a remainder hexamethyl derivative.

7. The medicinal agent according to claim 3, wherein the medicinal agent includes tri-p-aminotriphenylchloromethane in a form of a mixture, the pentamethyl and hexamethyl derivatives being at a ratio of 2-98 percent by volume pentamethyl derivative and a remainder hexamethyl derivative.

8. The medicinal agent according to claim 3, wherein the medicinal agent includes tri-p-aminotriphenylchloromethane in a form of a mixture, the tetramethyl, pentamethyl and hexamethyl derivatives being at a ratio of 2-98 percent by volume tetramethyl derivative, 2-98 percent by volume pentamethyl derivative and a remainder hexamethyl derivative.

9. A medicinal agent, which has an anti-blastomatous and immuno-modulating effect, comprising: the medicinal agent including a mixture of dextrin and pharmacologically representable derivatives of tri-p-aminotriphenylchloromethane in a form of either tetramethyl, pentamethyl or hexamethyl derivatives or a mixture of tetramethyl and pentamethyl derivatives or respectively a mixture of tetramethyl and hexamethyl derivatives or respectively a mixture of pentamethyl and hexamethyl derivatives or respectively a mixture of tetramethyl, pentamethyl and hexamethyl derivatives at a ratio of 10-95 percent by volume of the corresponding derivative or respectively a mixture of derivatives of the tri-p-aminotriphenylchloromethane and a remainder dextrin.

10. The medicinal agent according to claim 9, wherein a ratio of the mixture of tetramethyl and pentamethyl derivatives is 2-98 percent by volume tetra derivative and a remainder pentamethyl derivative.

11. The medicinal agent according to claim 9, wherein a ratio of the mixture of tetramethyl and hexamethyl derivatives is 2-98 percent by volume tetra derivative and a remainder hexamethyl derivative.

12. The medicinal agent according to claim 9, wherein a ratio of the mixture of pentamethyl and hexamethyl derivatives is 2-98 percent by volume penta derivative and a remainder hexamethyl derivative.

13. The medicinal agent according to claim 9, wherein a ratio of the mixture of tetra methyl, pentamethyl and hexamethyl derivatives is 2-98 percent by volume tetra derivative, 2-98 percent by volume penta derivative and the a remainder hexamethyl derivative.

14. The medicinal agent according to claim 1, wherein the medicinal agent includes tri-p-aminotriphenylchloromethane in a form of a mixture, the tetramethyl and pentamethyl derivatives being at a ratio of 2-98 percent by volume tetramethyl derivative and a remainder pentamethyl derivative.

15. The medicinal agent according to claim 1, wherein the medicinal agent includes tri-p-aminotriphenylchloromethane in a form of a mixture, the tetramethyl and hexamethyl derivatives being at a ratio of 2-98 percent by volume tetramethyl derivative and a remainder hexamethyl derivative.

16. The medicinal agent according to claim 1, wherein the medicinal agent includes tri-p-aminotriphenylchloromethane in a form of a mixture, the pentamethyl and hexamethyl derivatives being at a ratio of 2-98 percent by volume pentamethyl derivative and a remainder hexamethyl derivative.

17. The medicinal agent according to claim 1, wherein the medicinal agent includes tri-p-aminotriphenylchloromethane in a form of a mixture, the tetramethyl, pentamethyl and hexamethyl derivatives being at a ratio of 2-98 percent by volume tetramethyl derivative, 2-98 percent by volume pentamethyl derivative and a remainder hexamethyl derivative.
